(19) United States
(12) Patent Application Publication
(10) Pub. No.: US 2025/0002915 A1
(43) Pub. Date: Jan. 2, 2025

(54) MOLECULAR THERAPEUTICS FOR THE TREATMENT OF HYPERTROPHIC CARDIOMYOPATHY AND HEART FAILURE ASSOCIATED WITH MYBPC3 GENE MUTATIONS

(71) Applicant: University of Cincinnati, Cincinnati, OH (US)

(72) Inventor: Sakthivel Sadayappan, Blue Ash, OH (US)

(73) Assignee: University of Cincinnati, Cincinnati, OH (US)

(21) Appl. No.: 18/703,900

(22) PCT Filed: Oct. 25, 2022
(86) PCT No.: PCT/US2022/078650

§ 371 Date: Apr. 23, 2024

Related U.S. Application Data
(60) Provisional application No. 63/271,482, filed on Oct. 25, 2021.

(51) Int. Cl. C12N 15/113

(52) U.S. Cl. CPC C12N 15/113; C12N 2310/11; C12N 2310/313; C12N 2310/321

(57) ABSTRACT

Provided herein is an antisense oligonucleotide that specifically and selectively targets a mutant mRNA transcript of a cardiac myosin binding protein C (MYBPC3) allele having a 25 bp deletion (MYBPC3^{Δ25bp}) within intron 32. Also provided are pharmaceutical compositions comprising the antisense oligonucleotides and a method of treating a cardiac disorder associated with a 25 bp deletion of MYBPC3 via administration of an antisense oligonucleotide according to the present disclosure.

### CROSS-REFERENCE TO RELATED APPLICATION

This application claims priority to U.S. Provisional Application Ser. No. 63/271,482, filed Oct. 25, 2021, the entire contents of which are incorporated herein by reference.

### TECHNICAL FIELD

The present disclosure relates to the field of cardiac medicine. Specifically, the present disclosure relates to antisense oligonucleotides useful in the treatment of heart disease associated with cardiac myosin binding protein C (MYBPC3) gene mutations.

### SEQUENCE LISTING

A Sequence Listing XML having file name Sequence_Listing_CIN0351WO.xml, created on Oct. 19, 2022 (35,000 bytes), is incorporated herein by reference. The nucleic and amino acid sequences listed in the accompanying sequence listing are shown using standard abbreviations as defined in 37 C.F.R. 1.822.

### BACKGROUND

Mutations in the cardiac myosin binding protein C (MYBPC3) gene cause more than 40% of all known genetic hypertrophic cardiomyopathy. A common polymorphic variant MYBPC3 (˜4-6%) identified in 2001 is a 25-base pair (bp) deletion within intron 32 that results in a truncated carboxyl-terminus cMyBP-C protein with an altered amino acid sequence (hereinafter termed MYBPC3^{Δ25bp}). It is estimated that 100 million people carry the MYBPC3^{Δ25bp} variant, but unpredictable incomplete penetrance and expressivity presents challenges to determine the full impact on cardiovascular outcomes. Regardless, carriers of the MYBPC3^{Δ25bp} allele are at risk for left ventricular hypertrophy associated with sudden cardiac death (SCD) and progressive diastolic dysfunction leading to heart failure.

A need exists for improved compositions and methods for treating cardiomyopathy and heart failure associated with MYBPC3 gene mutations.

### SUMMARY

It has now been found that knockdown of mRNA transcripts from mutant cardiac myosin binding protein C (MYBPC3) alleles is effective in the treatment and prevention of cardiac disease associated with a 25 bp deletion in the C10 domain of c-MyBP-C protein. The 25 bp deletion results in exon 33 skipping or altered exon 33 splicing in the mutant mRNA transcript, which is associated with hypertrophic cardiomyopathy and heart failure in affected subjects.

Accordingly, in one embodiment, provided herein are antisense oligonucleotides (ASOs) that specifically and selectively target a mutant mRNA transcript of an MYBPC3 allele comprising a 25 bp deletion (MYBPC3^{Δ25bp}).

In another embodiment, provided herein is a method of treating a cardiac disorder in a subject in need thereof, the method comprising administering to the subject an effective amount of an ASO that targets a mutant mRNA transcript of an MYBPC3^{Δ25bp} allele.

In another embodiment, a pharmaceutical composition is provided, comprising an effective amount of an antisense oligonucleotide that specifically and selectively targets a mutant mRNA transcript of a MYBPC3 allele comprising a 25 bp deletion (MYBPC3^{Δ25bp}); and at least one pharmaceutically acceptable excipient.

In another embodiment, a method for modulating splicing of MYBPC3 processed mRNA in a cell is provided, the method comprising contacting the mRNA with an antisense oligonucleotide as set forth in Table 1 herein.

These and other objects, features, embodiments, and advantages will become apparent to those of ordinary skill in the art from a reading of the following detailed description and the appended claims.

### BRIEF DESCRIPTION OF THE DRAWINGS

FIG. 1 is a schematic diagram of hypertrophic cardiomyopathy (HCM) and dilated cardiomyopathy (DCM), compared to normal heart physiology.

FIG. 2A is a schematic diagram of wild type (WT) introns and exons of the 3′ region of MYBPC3 that codes for the C-terminal C9 and C10 domains of cMyBP-C.

FIG. 2B is a schematic diagram of MYBPC3^{Δ25bp} genotype, the variant being localized at the branch point in intron 32, which leads to exon 33 skipping, subsequent reading frame shift, and translation of a mutant cMyBP-C protein (cMyBP-CA10) with C10 domain modifications.

FIG. 2C shows an amino acid sequence alignment of WT and mutant C10 domains of cMyBP-C.

FIG. 2D is an illustration comparing the structures of WT and cMyBP-C^{Δ10}, wherein modifications to the C10 domain result in ablation of cMyBP-C^{Δ10} binding with the myosin LMM region.

FIG. 3A depicts PCR-based genotyping to determine WT, heterozygosity (A25 bp), and homozygosity of MYBPC3^{Δ25bp} variant carriers.

FIG. 3B depicts an echocardiography study showing the presence of increased left ventricular fractional shortening (LVFS %) (left panel) and normal left ventricular ejection fraction (right panel).

FIG. 3C depicts results of an exercise echocardiography and continuous cardiac monitoring showing that MYBPC3^{Δ25bp} is associated with LV hypercontraction under stress conditions with evidence of diastolic impairment.

FIG. 4A is a schematic diagram showing MYBPC3^{Δ25bp} maps to intron 32 between the splice branch point and polypyrimidine track. In the absence of MYBPC3^{Δ25bp} normal exon splicing occurs.

FIG. 4B shows that the presence of the MYBPC3^{Δ25bp} variant causes one of two possible changes (boxes), both resulting in the same partial deletion of the splice branch point and polypyrimidine track sequences.

FIG. 4C illustrates that the presence of additional risk factors may increase the potential for altered splicing and exon 33 skipping, leading to left ventricular hypertrophy (LVH) and heart failure (HF).

FIG. 5A illustrates normal splicing of the WT allele of MYBPC3.

FIG. 5B illustrates mutant splicing of MYBPC3^{Δ25bp} resulting in destruction of the branching point and potential involvement of nonsense-mediated decay (NMD) regulation to remove mutant mRNA lacking exon 33 in MYBPC3.

FIG. 6A is a schematic representation of cMyBP-C domain structure with its interacting partners, including myosin, actin, titin, and A-band in the sarcomere.

FIG. 6B depicts the intron and exon structure of the MYBPC3 gene.

FIG. 6C depicts normal splicing associated with an asymptomatic variant of MYBPC3^{Δ25bp}. Asymptomatic carriers with MYBPC3^{Δ25bp} variant show predominantly normal splicing.

FIG. 6D depicts splicing associated with a symptomatic variant of MYBPC3^{Δ25bp} resulting in exon 33 skipping and a frame shift resulting in incorrect splicing of exon 34. Symptomatic carriers with MYBPC3^{Δ25bp} variant show exon 33 splicing and the presence of LVH. HCM and HF phenotype.

FIG. 7A depicts graphs showing expression of cMyBP-C^{ΔC10} mRNA without exon 33 in culture of cardiomyocytes results in a significant decrease in contractile function without changes in calcium transients.

FIG. 7B depicts cross-sections of non-transgenic (NTG, wild type) and transgenic cMyBP-C^{ΔC10} mouse hearts, cMyBP-C^{ΔC10} mouse heart shows increased LV free wall and septal thickening at 3 months of age.

FIG. 7C depicts diastolic dysfunction was evidenced in cMyBP-C^{ΔC10mut} mice by m-mode echocardiography with increased ejection fraction (EF, %) and fractional shortening (FS, %).

FIG. 8A depicts an agarose gel image of RT-PCR demonstrating the presence of the altered splicing mRNA from MYBPC3^{Δ25bp} heterozygous mice, compared to non-transgenic mouse hearts.

FIG. 8B depicts results of DNA sequencing to determine the region of altered splicing in exon 33.

FIG. 8C is a schematic diagram of exon 33 normal splicing (left) and exon 33 altered splicing (right). Based on the altered splicing. ASOs (SS401 and SS501. Table 1) were designed to specifically target mutant mRNA.

FIG. 9A depicts ASO targeting strategy, wherein the regions of potential ASOs are marked as wild type mRNA, exon 33 skipped and exon 33 altered splicing MYBPC3 gene. ASOs are listed in Table 1. The designed ASOs are specific and selective to the exon 32/exon 34 junctions in the exon 33 skipped and exon 33 altered splicing mRNAs.

FIG. 9B depicts an overview of current and future research efforts, including human induced pluripotent stem cell-derived cardiomyocytes (hiPSC-CMs), preclinical mouse models, and clinical trials, leading effective therapies for treating MYBPC3^{Δ25bp} carriers for HCM and HF by administering the ASOs disclosed herein.

### DETAILED DESCRIPTION

The following description of particular embodiment(s) is merely exemplary in nature and is in no way intended to limit the scope of the invention, its application, or uses, which may vary. The invention is described with relation to the non-limiting definitions and terminology included herein.

The terminology used herein is for the purpose of describing particular embodiments only and is not intended to be limiting. As used herein, the singular forms “a,” “an.” and “the” are intended to include the plural forms, including “at least one.” unless the content clearly indicates otherwise. “Or” means “and/or.” As used herein, the term “and/or” includes any and all combinations of one or more of the associated listed items. It will be further understood that the terms “comprises” and/or “comprising.” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise indicated, all numbers expressing quantities of components, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about.” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all terms (including technical and scientific terms) used herein have the same meaning as commonly understood by one of ordinary skill in the art to which this disclosure belongs. It will be further understood that terms such as those defined in commonly used dictionaries should be interpreted as having a meaning that is consistent with their meaning in the context of the relevant art and the present disclosure, and will not be interpreted in an idealized or overly formal sense unless expressly so defined herein.

The term “subject,” as used herein, refers to any mammalian subject, including mice, rats, rabbits, pigs, monkeys, humans, and the like. In a specific embodiment, the subject is a human.

The terms “treat,” “treatment,” and “treating,” as used herein, refer to a method of alleviating or abrogating a disease, disorder, and/or symptoms thereof. In a specific embodiment, the disease or disorder is heart failure. In a another specific embodiment, the disease or disorder is hypertrophic cardiomyopathy.

An “antisense oligonucleotide” or “ASO,” as used herein, refers to a small (about 18-30 nucleotides) single-stranded deoxyribonucleotide that binds a target mRNA through Watson-Crick base pairing and modulates gene expression, for example, via modulating mRNA translation. Typically, downregulation of the molecular target occurs via induction of RNase H endonuclease activity, which cleaves the RNA-DNA heteroduplex. ASOs may also modulate or downregulate the molecular target by inhibiting formation of a 5′ cap, splice-switching, and steric hindrance of ribosomes to block translation. See Watts and Corey, Gene silencing by siRNAs and antisense oligonucleotides in the laboratory and the clinic. J. Pathol . . . 226 (2): 365-79 (2012); Roberts, et al., Advances in oligonucleotide drug delivery. Nat. Rev. Drug Discov. 19 (10): 67-94 (2020).

As used herein, the term “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

The present disclosure is related to treatment of cardiac dysfunction caused by a polymorphic 25-base pair (bp) deletion within intron 32 (Δ25 bp) of the MYBPC3 gene. This variant is present in an estimated 6% of South Asian individuals and predisposes a carrier to multiple forms of cardiomyopathy including late onset left ventricular dysfunction, hypertrophic cardiomyopathy, heart failure (HF) and sudden cardiac death (SCD) with an odds ratio of symptomatic disease as high as 7 depending on the presence of other co-morbidities.

Provided herein are compositions and methods useful in the treatment and prevention of cardiac disease associated with a C10 domain mutation of c-MyBP-C protein. Specifically, described herein are ASOs that specifically and selectively target a mutant mRNA transcript of an MYBPC3 allele comprising a 25 bp deletion (MYBPC3^{Δ25bp}). The ASOs disclosed herein selectively knock down mutant mRNA transcripts of MYBPC3^{Δ25bp}, wherein the mutant transcripts skip exon 33 of MYBPC3, or wherein the mutant transcripts are a product of altered splicing of exon 33 of MYBPC3. ASOs disclosed herein target one or more of (i) the junction of exon 32/exon 34 of MYBPC3, or (ii) the junction of exon 32/exon 33 of MYBPC3 with altered splicing. Pharmaceutical compositions and methods for treating MYBPC3^{Δ25bp}-mediated heart failure by administering the ASOs of the disclosure are also provided.

### Inherited Cardiomyopathies

Cardiomyopathies are a class of heterogeneous cardiac diseases that contribute to the development of heart failure, a complex clinical phenotype that poses a major worldwide public health problem. The statistics are daunting: associated heart failure alone affects about 4.5 million patients with frequent hospitalization and mortality of 300,000 deaths each year. Cardiomyopathy comprises four distinct disorders: arrhythmogenic right ventricular cardiomyopathy (ARVC), hypertrophic cardiomyopathy (HCM), dilated cardiomyopathy (DCM), and restrictive cardiomyopathy. Combined, these disorders affect an estimated 600,000 people in the United States and 14.25 million people worldwide. Clinical studies of cardiomyopathies show that about 50% of the cases are inherited, with the remaining fraction being either unexplained, secondary to epigenetic and/or somatic mutations, or secondary primarily to other vascular dysfunction, such as outflow obstruction. With advances in DNA sequencing approaches, some etiological mutations of HCM have increasingly been identified by screening candidate genes.

### Clinical and Pathological Features of HCM

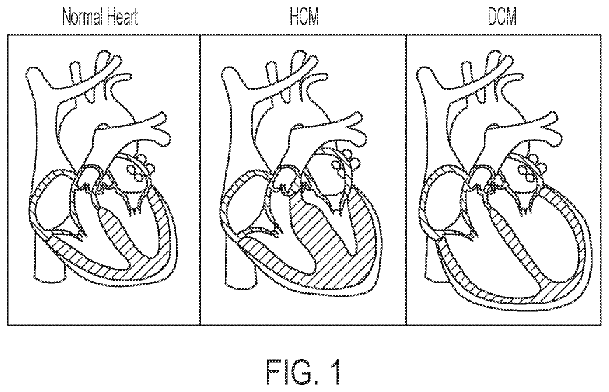

HCM is characterized by an asymmetric increase in left ventricular cardiac muscle mass in the absence of dilatation (FIG. 1). Diagnosis is made by two-dimensional echocardiography, often because of a positive family history, but in many cases due to an incidental finding. While HCM may remain a stable structural cardiac finding. HCM may unpredictably progress from minor left ventricular function appreciated under formal stress testing to hypercontractility, reduced exercise tolerance, and HF. Sudden cardiac death (SCD) may occur 1% of all patients at any age secondary to ventricular fibrillation. A family history with repeated cases of premature sudden death is often elicited in retrospect and, for unclear reasons, SCD occurs tragically in athletes that are ostensibly otherwise healthy.

Concentric hypertrophy is prominent in the left ventricle and frequently affects the septum between the ventricles. The free and posterior wall can also be increased in mass, sometimes restricted to the apical region. Increase in mass in a region close to the left ventricular outflow tract may obstruct normal blood flow into the aorta. Moderate wall thickness appears to be well-tolerated, but high values may constitute a major risk of cardiac death. SCD has been reported in individuals carrying mutations classically associated with HCM, even in the absence of significant ventricular wall thickening.

Distinctive histological changes in the myocardium are fibrosis and cytological disarray. Gross disorganization of cardiomyocytes is seen, together with interstitial fibrosis, resulting in a characteristic whorled array. Myofilaments within cells may lose their normal parallel alignment, evident in electron microscopy. Foci of disorganized cells are often interspersed among areas of hypertrophied muscle, otherwise normal in appearance. These changes are also seen in normal heart tissue, but if extensive, may contribute to symptoms by a loss of distensibility of the myocardium and impaired diastole, or by confounding regular conduction, which may cause arrhythmia. Early on. HCM is associated with hyperdynamic systolic function. Early, rapid and near-complete emptying of the ventricle is presumably related to increased muscle mass, concomitant changes in ventricular geometry or alterations in cross-bridge cycling of myofilaments. Altered handling of calcium is suspected to contribute to pathogenesis.

Mutations in 8 sarcomeric genes (β-myosin heavy chain (MY117), titin (TTN), cardiac myosin-binding protein C (MYPBC3), cardiac troponin T (TNNT2), α-tropomyosin (TPM1), cardiac troponin I (TNNI3), cardiac actin (ACTC), regulatory myosin light chain (MYL2) and essential myosin light chain (MYL3)) account for about 80% of all index cases of HCM. These genes code for proteins that contribute to motility and force development of cardiac muscle, either directly by forming reversible cross-bridges between myosin and actin filaments, or indirectly by regulating this process. Mutations in MYH7 and MYPBC3 occur most often and account for approximately 80% of HCM cases, while mutations in TNNT2, TNNI3, ACTC, TPM1, MYL3 and MYL2 collectively account for less than 20% of HCM cases.

Mutated genes associated with a clinical phenotype typically affect evolutionarily conserved protein regions. Most of the mutational amino acid exchanges induce an altered electrochemical charge in the protein expected to have a major impact on protein folding and function. Either homozygosity or the presence of heterozygotic mutations in two different genes is associated with a more severe phenotype compared to heterozygosity found in carriers with only one mutated allele. In vitro analysis of β-myosin heavy chains and other contractile proteins has demonstrated altered function with respect to contraction, force development, and ATP hydrolysis typical of HCM. Importantly, mutations in humans associated with HCM have been demonstrated in transgenic animal models reproduce various cardiac dysfunctions similar to those seen in these patients.

The molecular mechanisms underlying the relationship between certain mutations and HCM have not been fully understood. In most cases, research has focused on probing the biochemical consequences of a mutation on altered proteins and myocardium performance. Other focus is understanding the mechanism leading to compensatory changes in the heart, and in particular hypertrophy of cardiac muscle cells. Secondary structural alterations (fibrosis, changes in the media of small vessels) contribute to clinical conditions and represent another area of investigation.

In vitro studies, as well as results obtained with genetically manipulated animals (predominantly mice), have provided some insight into the pathogenesis of human HCM. Experimental findings and epidemiological and clinical data suggest that mutations differ with respect to their mode of action and phenotypic consequences. Mutations in the cardiac troponin T gene tend to cause severe disease with early onset in life and a relatively high risk of sudden cardiac death, typically without marked hypertrophy. Mutations in the MYH7 gene cover a broad spectrum of clinical presentations, ranging from severe to mild disease courses. A missense mutation in codon 403 (replacement of arginine by glutamine) of this gene carries a particularly high risk of premature sudden death for carriers. While not desiring to be bound by theory, evidence suggests that a secondary increase of toxic proteins in the heart contributes to the etiology of HF. Thus, treating proteotoxicity represents a therapeutic approach to reduce accumulation of toxic proteins and/or diminish toxic effects.

Impaired energy balance and dysfunctional contractility are also implicated in HCM pathogenesis. Myoblasts are committed but undifferentiated muscle stem cells readily induced to form contracting myofibers in vitro. Mutations comprising missense exchanges in two positions known to cause disease in humans (codon Ile79Asn, and Arg92Gln) have been previously explored. Studies indicate the presence of increased calcium sensitivity of force development, an accelerated dissociation of cross-bridges, and a decreased production of force. These data suggest that less force per ATP (or energy invested per cross-bridge cycle) was produced. Thus, the overall cost of energy to maintain normal function may be higher in hearts with these mutations than in wild type hearts.

### MYBPC3 Gene Mutations

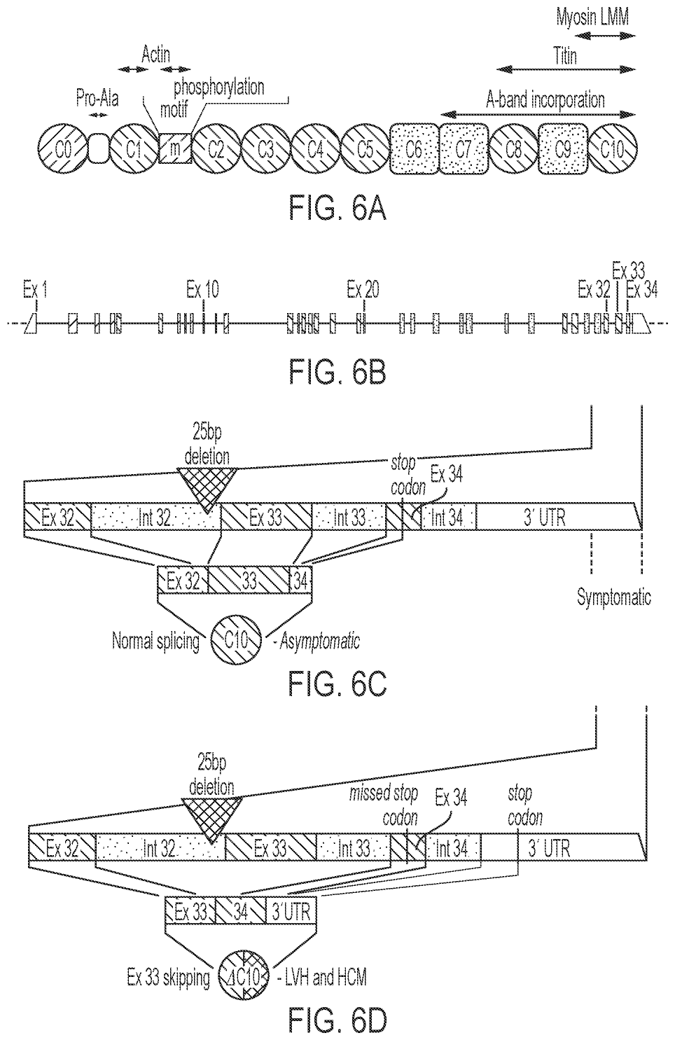

MYBPC3 encodes cardiac myosin binding protein-C (cMyBP-C) and is associated with ˜40% of all HCM cases. MYBPC3 comprises 24 kb of genomic DNA with 35 exons that encode a protein of 1.274 amino acids. Most mutations are insertions, deletions, or splice site mutations that result in truncation of the cMyBP-C protein with loss of the myosin and titin binding sites. Missense mutations that preserve the myosin and titin binding sites have also been reported. Predominantly, mutations in cMyBP-C affect the structure of motifs, thereby altering function. cMyBP-C interacts with actin via the C1 and M domains and with myosin via the C1. M. and C2 domains. A proline-rich region, located between the C0 and C1 domains, interacts with a-tropomyosin (α-TM) (see FIG. 6A).

Both actin and myosin interactions are regulated by M-domain phosphorylation. The N′-region acts a molecular brake (ON/OFF state) controlling actomyosin regulation and modulating sarcomere structure and function. Clinically, when cMyBP-C is proteolyzed during ischemia injury and heart failure, the N′-region, consisting of the C0, C1, and 17 residues of the M domain (i.e., C0-C1f), is cleaved from the full-length protein. The phosphorylation-dependent binding of domains C0-C2 to S2 of myosin and actin are well-established. Domains C7 to C10 of the C-terminal region of cMyBP-C bind the backbone region of the myosin thick filament. The primary myosin and titin binding regions of cMyBP-C are localized to domains C10 and domain C8-C10, respectively. Studies have shown that C-terminal domains, C8-C10, are the minimal requirement for incorporation into the A-band of the sarcomere, with domain C7 improving the targeting of cMyBP-C to the C-Zone.

The majority of HCM mutations result in premature termination of translation of the C-terminus of cMyBP-C, thus eliminating the titin and/or myosin binding sites. While not desiring to be bound by theory, it is believed that this results in minimal or nil incorporation of these truncated mutant proteins into the sarcomere.

The compositions and methods of the present disclosure relate to a polymorphic deletion variant (MYBPC3^{Δ25bp}) that modifies the C-terminus of cMyBP-C. Nine key residues involved in binding the C-terminal C10 domain to the filament have been identified by mutagenesis/sedimentation assays. When these residues are positioned onto a model of domain C10, the myosin binding faces are located on two surfaces. Thus, the C10 domain regulates cMyBP-C localization, incorporation, and thick filament alignment in the sarcomere. Alterations in the C10 domain may result in removal of mutant cMyBP-C from the sarcomere, as well as changes in the thick filament structure and function, leading to the development of HCM. The presence of MYBPC3^{Δ25bp} affects the C10 domain and causes HCM and HF.

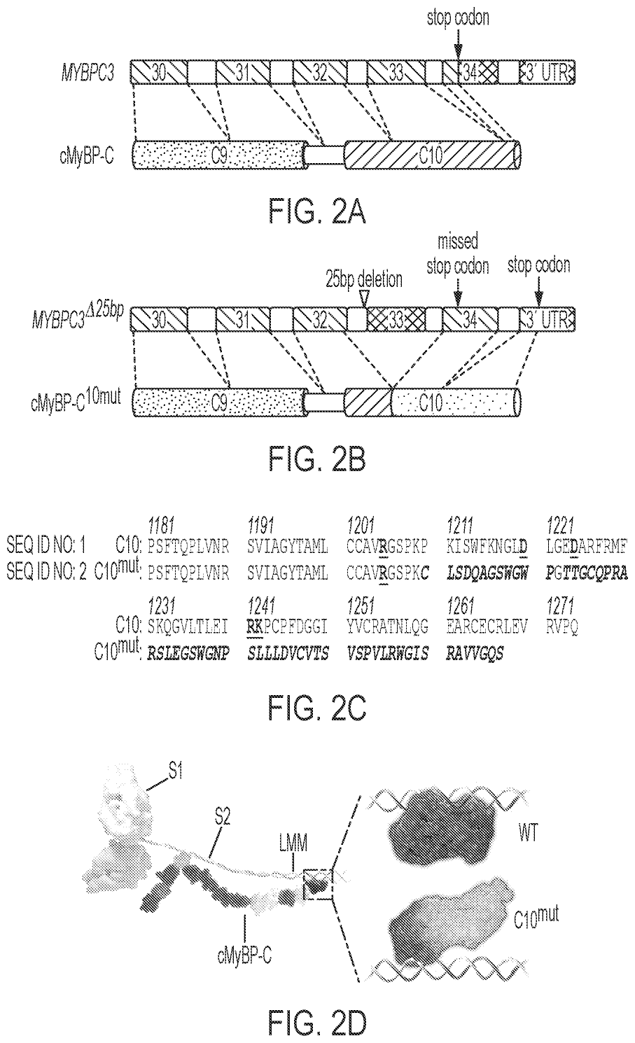

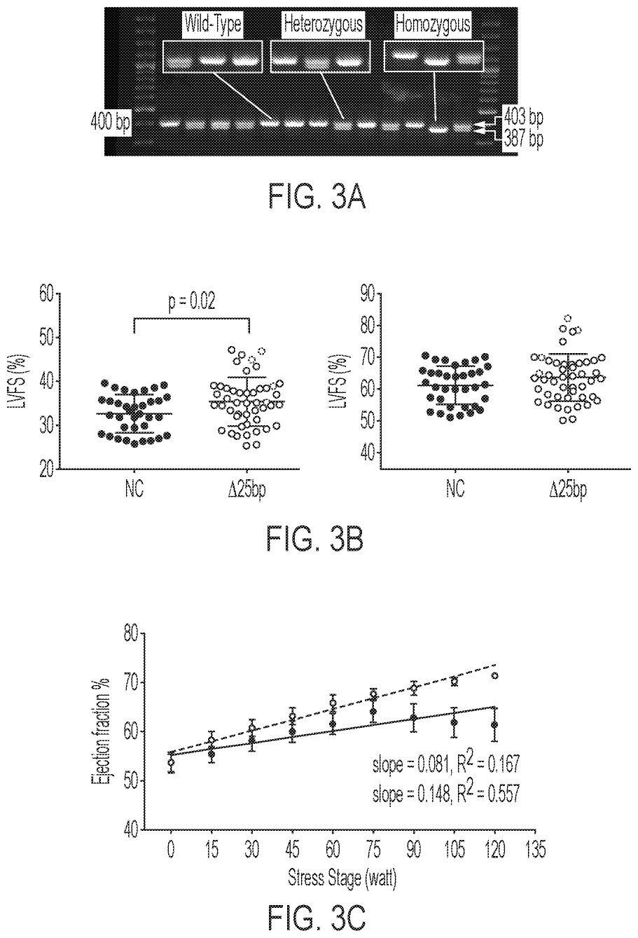

A 25-base pair (bp) deletion in the MYBPC3 gene within intron 32 alters the carboxyl-terminus of cMyBP-C and produces a truncated protein with an altered amino acid sequence (MYBPC3^{Δ25bp}) denoted as the cMyBP-C^{ΔC10} protein (FIG. 2A-2D). The MYBPC3^{Δ25bp} variant can be easily genotyped by a PCR-based assay (FIG. 3A) and fluorescence based real-time PCR-based assays. It is estimated that 100 million people of South Asian descent carry the MYBPC3^{Δ25bp} variant, which can be traced to a mutation that occurred approximately 40 thousand years ago. Although common, 6% of South Asian subjects in the United States tested positive for the MYBPC3^{Δ25bp} variant. Incomplete penetrance and expressivity markedly reduces the incidence of clinical morbidity and mortality for most carriers. MYBPC3^{Δ25bp} variant is associated with a range of phenotypes, from asymptomatic normal hearts to diastolic dysfunction, HCM, DCM, and HF. Symptomatic carriers may experience contractile dysfunction at baseline (FIG. 3B). The relative risk of developing HCM among mutant carriers is 5.3-fold and 6.99-fold among independent subjects with HCM.

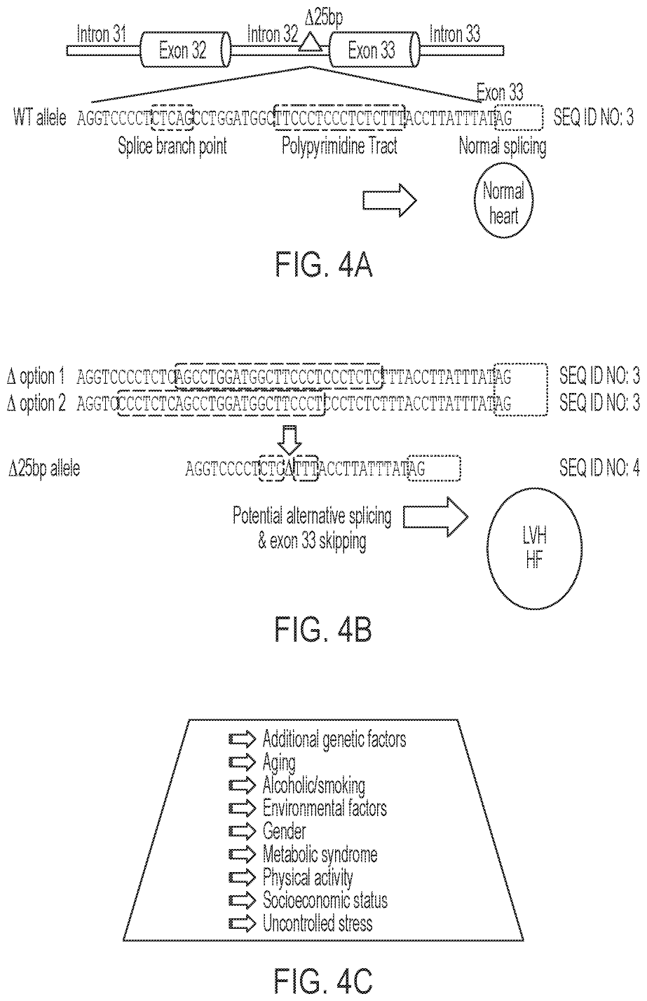

Additional modifiers, including additional genetic mutations, contribute to the development and manifestation of the cardiomyopathy phenotype, indicating that a more complex genetic architecture is involved in the disease (FIG. 4A-4C). If carriers of MYBPC3^{Δ25bp} also carry a mutation in the β-myosin heavy chain, this combination frequently results in sudden cardiac death. Seventy percent of all MYBPC3 mutations, including MYBPC3^{Δ25bp}, are predicted to produce C′-truncated proteins that lack key myosin-binding residues, resulting in altered sarcomere structure, reduced cardiac contractility, and the HCM phenotype. However, the pathogenic process for each C′ truncation mutation is unique, resulting in variable phenotypes. Additional genetic variants in the presence of MYBPC3^{Δ25bp} variant contribute to the development of cardiomyopathy and HF in MYBPC3^{Δ25bp} variant carriers.

Complementary DNA sequencing of mRNA isolated from the biopsy of a MYBPC3^{Δ25bp}-positive patient confirmed the presence of exon 33 skipping in vivo. Exon 33 skipping results in the loss of 62 amino acids and production of a modified C10 domain of cMyBP-C. Exon 33 skipping also moves the stop codon to the 3′-untranslated region (UTR) and adds 55 amino acids in the C10 domain at the carboxyl terminus (cMyBP-C^{ΔC10}) (FIG. 6C-6D). If the C10 domain is modified or truncated, cMyBP-C will not localize in the sarcomere. The presence of the MYBPC3^{Δ25bp} variant correlates with exon 33 skipping during the transcription process.

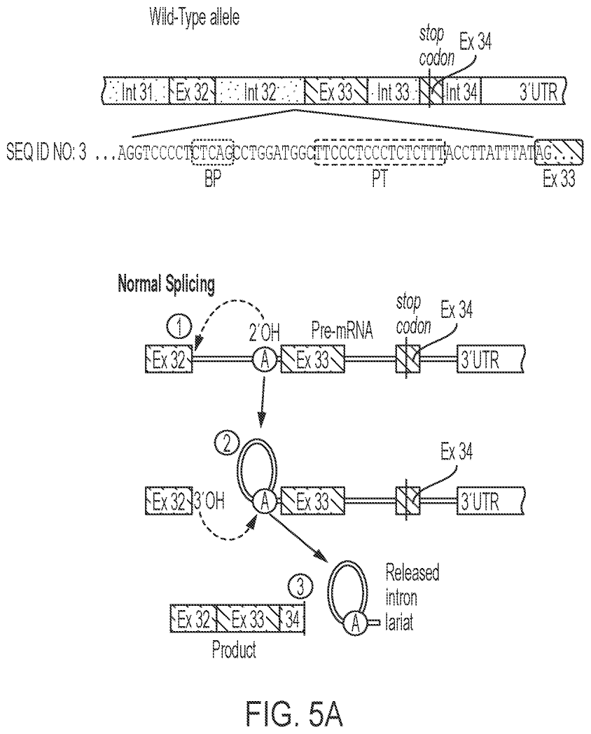

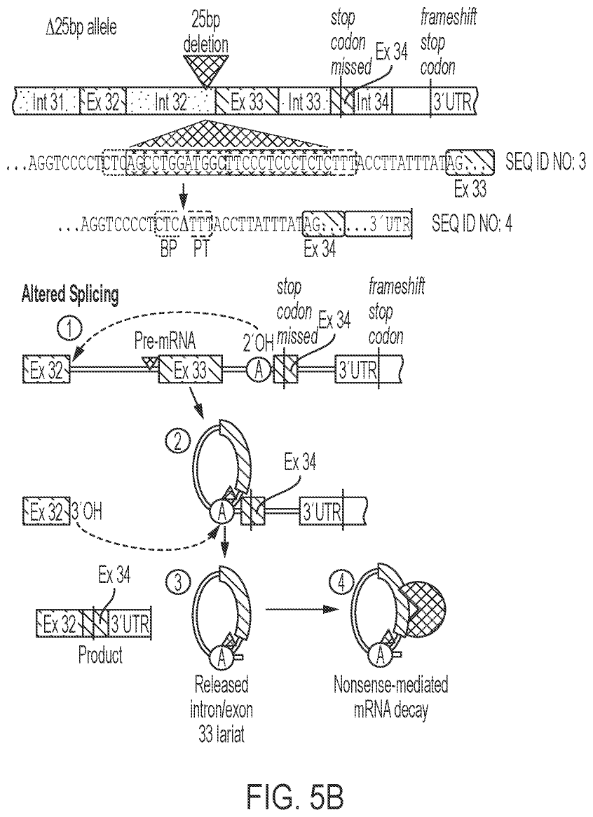

The MYBPC3^{Δ25bp} variant in the presence of exon 33 skipping is pathogenic, both in vitro and in vivo. While not desiring to be bound by theory, it is believed that the presence of MYBPC3^{Δ25bp} causes variable splicing in the intronic/exonic regions around the 25 bp deletion and that such splicing allows RNA polymerase to, at least partially, skip exon 33 in MYBPC3^{Δ25bp} carriers. Upon expression of cMyBP-C^{ΔC10}, the underlying molecular mechanism could be attributed to haploinsufficiency, a poison polypeptide effect, or activation of unfolded protein responses. Under baseline conditions without any secondary metabolic cardiovascular stress, it is expected that the nonsense-mediated mRNA decay (NMD) process is activated, resulting efficient removal of the mutant mRNA (FIG. 5B). However, in the presence of secondary metabolic cardiovascular stress, NMD is dysfunctional, leading to accumulation of mutant mRNA and protein and causing poison polypeptide effects and HCM and HF phenotypes (FIG. 6D). It is thus believed that the degree by which the heterozygous MYBPC3^{Δ25bp} variant leads to disease reflects how the individual cell processes the mutant RNA.

Carriers of MYBPC3^{Δ25bp} often have mutations in other sarcomeric proteins, resulting in poorer prognoses. For example, if carriers of MYBPC3^{Δ25bp} also carry a mutation in the β-myosin heavy chain, the combination is commonly associated sudden cardiac death. Particularly concerning statistics indicate that South Asians carrying this mutation have 50% higher rates of morbidity and mortality after ischemia-reperfusion injury compared with other ethnic groups. Previous studies have shown that the presence of MYBPC3^{Δ25bp} leads to higher susceptibility to coronary artery disease, suggesting that MYBPC3^{Δ25bp} pathogenicity can exacerbate cardiac dysfunction in patients with myocardial infarction. Early identification and intervention in MYBPC3^{Δ25bp} carriers is likely to have an impact on a variety of cardiovascular disease outcomes.

### Antisense Oligonucleotides

Provided herein are selective ASOs against MYBPC3^{Δ25bp} variants that have been designed to target MYBPC3^{Δ25bp} and downregulate expression thereof.

In one embodiment, an antisense oligonucleotide is provided that specifically and selectively targets a mutant mRNA transcript of a cardiac myosin binding protein C (MYBPC3) allele comprising a 25 bp deletion (MYBPC3^{Δ25bp}). In embodiments, the MYBPC3^{Δ25bp} allele comprises a 25 bp deletion within intron 32 of MYBPC3. In embodiments, the MYBPC3^{Δ25bp} allele comprises the sequence AGGTCCCCTCTCTTTACCTTATTTATAG (SEQ ID NO: 4) or a sequence having at least 80%, 85%, 90%, 95%, 96%, 97%, 98%, 99%, or 100% identity therewith.

In embodiments, the 25 bp deletion results in production of a target mutant mRNA transcript of the MYBPC3^{Δ25bp} allele, wherein exon 33 of MYBPC3 is skipped. i.e., not present in the transcript. In other embodiments, the 25 bp deletion results in production of a target mutant mRNA transcript of the MYBPC3^{Δ25bp} allele, wherein the target mutant mRNA transcript is a product of altered splicing of exon 33 of MYBPC3. In certain embodiments, the target mutant mRNA transcript of the MYBPC3^{Δ25bp} allele comprises a mis-spliced full-length exon 34 of MYBPC3. In certain embodiments, the mutant mRNA transcript of the MYBPC3^{Δ25bp} allele comprises at least a portion of the 3′ untranslated region of MYBPC3.

In embodiments, the ASO targets a mutant mRNA transcript of the MYBPC3^{Δ25bp} allele that encodes a cMyBP-C protein comprising a mutant C10 domain (cMyBP-C^{C10mut}).

In embodiments, provided herein are ASOs that hybridize to SEQ ID NO: 4 or GCTATAATGCCATCCTCTGCTGTGCTGTCCGAGGTAGTCCTAAGGGCCACCAACTTG CAGGGCGAGGCACAGTGTGAGTGCCGCCTGGAGGTGCGAGTTCCTCAGT (SEQ ID NO: 5), wherein SEQ ID NO: 5 corresponds to a MYBPC3^{Δ25bp} mRNA transcript comprising altered splicing of exon 33. In embodiments, the ASOs hybridize to SEQ ID NO: 4 or SEQ ID NO: 5 under stringent conditions.

In embodiments, an ASO according to the present disclosure has at least 70%, at least 75%, at least 80%, at least 85%, at least 90%, at least 95%, at least 96%, at least 97%, at least 98%, at least 99%, or 100% sequence identity with ACTGAGGCACTTGGGGTAC (SEQ ID NO: 12), CACTGAGGCACTTGGGGCTA (SEQ ID NO: 13), TCACTGAGGCACTTGGGGCTA (SEQ ID NO: 14), GTCACTGAGGCACTTGGG (SEQ ID NO: 15), ACTGAGGAACTTAGGACTA (SEQ ID NO: 19). CACTGAGGAACTTAGGACTA (SEQ ID NO: 20), TCACTGAGGAACTTAGGACT (SEQ ID NO: 21), AAGTTGGTGGCCCTTGGGGC (SEQ ID NO: 22), AGTTGGTGGCCCTTGGGGCT (SEQ ID NO: 23), GTTGGTGGCCCTTGGGGCTA (SEQ ID NO: 24), GTTGGTGGCCCTTAGGACTA (SEQ ID NO: 25), AGTTGGTGGCCCTTATTAC (SEQ ID NO: 26), AAGTTGGGTGGCCCTTAGGAC (SEQ ID NO: 27), of any of the sequences set forth in Table 1.

In another embodiment, an ASO according to the present disclosure is complementary to a sequence having at least 70%, at least 75%, at least 80%, at least 85%, at least 90%, at least 95%, at least 96%, at least 97%, at least 98%, at least 99%, or 100% sequence identity with SEQ ID NO: 12, SEQ ID NO: 13, SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or any of the sequences set forth in Table 1.

In embodiments, the ASOs targeting MYBPC3^{Δ25bp} variant-specific exon skipping (exon 33 skipping) are selected from SEQ ID NOs: 12-15 (human Δexon 33) and SEQ ID NOs: 19-21 (mouse Δexon 33).

In embodiments, the ASOs targeting MYBPC3^{Δ25bp} variant-specific altered splicing of exon 33 are selected from SEQ ID NOs: 22-24 (human Δexon 33 altered splicing) and SEQ ID NOs: 25-27 (mouse Δexon 33 altered splicing).

Optionally. ASOs as described herein may comprise posttranslational, genetic, and/or epigenetic modifications.

As used herein, the term “2′-modified” or “2′-substituted” refers to a sugar comprising substituent at the 2′ position other than H or OH. 2′-modified monomers, include, but are not limited to, monomers (e.g., nucleosides and nucleotides) with 2′-substituents, such as allyl, amino, azido, thio, O-allyl, O—C₁-C₁₀ alkyl, —OCF₃, O—(CH₂) 2-O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. Suitable 2′ modifications are known in the art. See, e.g., U.S. Pat. No. 10,493,092, incorporated herein by reference. In specific embodiments, the 2′-modification(s) are independently selected from 2′-O-methyl. 2′-O-methoxyethyl (2-MOE), or 2′-fluoroarabinonucleic acid.

In embodiments, the ASOs described herein are synthetically modified to increase potency.

As used herein, the term “gapmer” refers to a chimeric oligomeric compound comprising a central region (a “gap”) and a region on either side of the central region (the “wings”), wherein the gap comprises at least one modification difference compared to each wing. Such modifications include nucleobase, monomeric linkage, and sugar modifications as well as the absence of modification (unmodified RNA or DNA). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in each of the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) within each wing are the same. In certain embodiments, the modification(s) in one wing are different from the modification(s) in the other wing.

In embodiments, such gapmer oligonucleotides may contain up to about 1, 2, 3, 4, 5, 6, or 7 chemically modified nucleotide sugars at each flanking region. Such modifications may be independently selected and include, but are not limited to, 2′-O-methyl, 2′-O-methoxyethyl, or 2′-fluoroarabinonucleic acid on each terminus flanking a central 8, 9, 10, 11, 12, 13, 14, 15, 16, 17, or 18 nucleotide base “gap” of DNA. The chemically modified nucleotides increase nuclease resistance and increase affinity of the ASO for target sequences, while the unmodified gap region permits formation of the DNA: RNA heteroduplex that provides a substrate for RNase H.

In embodiments, the ASOs comprise modifications to the oligonucleotide backbone and/or sugar substitutions. Such modifications include, but are not limited to, morpholino substitutions, phosphorodiamidate linkages, phosphorothioate (PS) linkages, and the like.

In specific embodiments, nucleotide modifications are selected from morpholino phosphorothioates (2′MOE/PS;

/52MOErG/*/i2MOErA/*/i2MOErA/*/i2MOErA/*/i2MOErT/
*C*T*T*G*G*G*C*T*T*G*/i2
MOErG/*/i2MOErG/*/i2MOErG/*/i2MOErC/*/32MOErT/)
or phosphorodiamidate
(2′MOE/phosphodiester/PMO;
52MOErA//i2MOErG//i2MOErG//i2MOErA//i2MOErA/
ATCTTGGGCT/i2MOErT//i2MOErG//i2MOErG//i2MOErG//
32MOErG).

Exemplary ASOs according to the present disclosure are set forth in Table 1.

TABLE 1
Antisense Oligonucleotides Targeting WT and Mutant mRNA of MYBPC3

| SEQ | | | | | | |
|---|---|---|---|---|---|---|
| ID | | | Posi- | | | |
| NO | Label | Description | tion | Allele | ASO Sequence | Optional Modifications |
| 8 | SS0001A | Human WT | Exon | WT | AGGAAATCTTGGGCTTGGGG | 2′ MOE/phosphodiester |
| | | | 32/Exon | | | and/or 2′ MOE/PS |
| | | | 33 | | | |
| 9 | SS0001B | Human WT | Exon | WT | GAAATCTTGGGCTTGGGGCT | 2′ MOE/phosphodiester |
| | | | 32/Exon | | | and/or 2′ MOE/PS |
| | | | 33 | | | |
| 10 | SS0001C | Human WT | Exon | WT | AATCTTGGGCTTGGGGCTAC | 2′ MOE/phosphodiester |
| | | | 32/Exon | | | and/or 2′ MOE/PS |
| | | | 33 | | | |
| 11 | SS0001D | Human WT | Exon | WT | ATCTTGGGCTTGGGGCTA | 2′ MOE/phosphodiester |
| | | | 32/Exon | | | and/or 2′ MOE/PS |
| | | | 33 | | | |
| 12 | SS101A | Human ΔExon | Exon | Mutant | ACTGAGGCACTTGGGGTAC | 2′ MOE/phosphodiester |
| | | 33 | 32/Exon | | | and/or 2′ MOE/PS |
| | | | 34 | | | |
| 13 | SS101B | Human ΔExon | 32/Exon | Mutant | CACTGAGGCACTTGGGGCTA | 2′ MOE/phosphodiester |
| | | 33 | Exon 34 | | | and/or 2′ MOE/PS |
| 14 | SS101C | Human ΔExon | Exon | Mutant | TCACTGAGGCACTTGGGGCT | 2′ MOE/phosphodiester |
| | | 33 | 32/Exon | | | and/or 2′ MOE/PS |
| | | | 34 | | | |
| 15 | SS101D | Human ΔExon | Exon | Mutant | GTCACTGAGGCACTTGGG | 2′ MOE/phosphodiester |
| | | 33 | 32/Exon | | | and/or 2′ MOE/PS |
| | | | 34 | | | |
| 16 | SS201A | Mouse WT | Exon | WT | GAAATCTTGGGCTTAGGACT | 2′ MOE/phosphodiester |
| | | | 32/Exon | | | and/or 2′ MOE/PS |
| | | | 33 | | | |
| 17 | SS201B | Mouse WT | Exon | WT | AAATCTTGGGCTTAGGACTA | 2′ MOE/phosphodiester |
| | | | 32/Exon | | | and/or 2′ MOE/PS |
| | | | 33 | | | |
| 18 | SS201C | Mouse WT | Exon | WT | AATCTTGGGCTTAGGACTAC | 2′ MOE/phosphodiester |
| | | | 32/Exon | | | and/or 2′ MOE/PS |
| | | | 33 | | | |
| 19 | SS301A | Mouse ΔExon | Exon | Mutant | ACTGAGGAACTTAGGACTAC | 2′ MOE/phosphodiester |
| | | 33 | 32/Exon | | | and/or 2′ MOE/PS |
| | | | 34 | | | |
| 20 | SS301B | Mouse ΔExon | Exon | Mutant | CACTGAGGAACTTAGGACTA | 2′ MOE/phosphodiester |
| | | 33 | 32/Exon | | | and/or 2′ MOE/PS |
| | | | 34 | | | |
| 21 | SS301C | Mouse ΔExon | Exon | Mutant | TCACTGAGGAACTTAGGACT | 2′ MOE/phosphodiester |
| | | 33 | 32/Exon | | | and/or 2′ MOE/PS |
| | | | 34 | | | |
| 22 | SS401A | Human ΔExon | Exon | Mutant | AAGTTGGTGGCCCTTGGGGC | 2′ MOE/phosphodiester |
| | | 33 alt splice | 32/Exon | | | and/or 2′ MOE/PS |
| | | | 33 alt | | | |
| | | | splice | | | |
| 23 | SS401B | Human ΔExon | Exon | Mutant | AGTTGGTGGCCCTTGGGGCT | 2′ MOE/phosphodiester |
| | | 33 alt splice | 32/Exon | | | and/or 2′ MOE/PS |
| | | | 33 alt | | | |
| | | | splice | | | |
| 24 | SS401C | Human ΔExon | Exon | Mutant | GTTGGTGGCCCTTGGGGCTA | 2′ MOE/phosphodiester |
| | | 33 alt splice | 32/Exon | | | and/or 2′ MOE/PS |
| | | | 33 alt | | | |
| | | | splice | | | |
| 25 | SS501A | Mouse ΔExon | Exon | Mutant | GTTGGTGGCCCTTAGGACTA | 2′ MOE/phosphodiester |
| | | 33 alt splice | 32/Exon | | | and/or 2′ MOE/PS |
| | | | 33 alt | | | |
| | | | splice | | | |
| 26 | SS501B | Mouse ΔExon | Exon | Mutant | AGTTGGTGGCCCTTAGGACT | 2′ MOE/phosphodiester |
| | | 33 alt splice | 32/Exon | | | and/or 2′ MOE/PS |
| | | | 33 alt | | | |
| | | | splice | | | |
| 27 | SS501C | Mouse ΔExon | Exon | Mutant | AAGTTGGTGGCCCTTAGGAC | 2′ MOE/phosphodiester |
| | | 33 alt splice | 32/Exon | | | and/or 2′ MOE/PS |
| | | | 33 alt | | | |
| | | | splice | | | |

Table 2 sets forth sequences for the design of ASOs according to the present disclosure, wherein underlined portions of the sequences designate target junctions between exons suitable for designing ASOs.

TABLE 2
Sequences for Designing ASOs that Target MYBPC3 and MYBPC3^{Δ25bp}

| SEQ | | | | |
|---|---|---|---|---|
| ID NO | Label | Name | Sequence (5′-3′) | Position |
| 28 | SS001 | Human WT | GAGTGGTTCACCGTCTTGGAGCATTACCG | Exon 31/ |
| | | | CCGCACCCACTGCGTGGTGCCAGAGCTCA | Exon 32/ |
| | | | TCATTGGCAATGGCTACTACTTCCGCGTCT | Exon 33/ |
| | | | TCAGCCAGAATATGGTTGGCTTTAGTGAC | Exon 34/ |
| | | | AGAGCGGCCACCACCAAGGAGCCCGTCTT | Exon 35 |
| | | | TATCCCCAGACCAGGCATCACCTATGAG | |
| | | | CCACCCAACTATAAGGCCCTGGACTTC | |
| | | | TCCGAGGCCCCAAGCTTCACCCAGCCC | |
| | | | CTGGTGAACCGCTCGGTCATCGCGGGC | |
| | | | TACACTGCTATGCTCTGCTGTGCTGTCC | |
| | | | GGGGTAGCCCCAAGCCCAAGATTTCCTG | |
| | | | GTTCAAGAATGGCCTGGACCTGGGAGAA | |
| | | | GACGCCCGCTTCCGCATGTTCAGCAAGCA | |
| | | | GGGAGTGTTGACTCTGGAGATTAGAAAGC | |
| | | | CCTGCCCCTTTGACGGGGGCATCTATGTC | |
| | | | TGCAGGGCCACCAACTTACAGGGCGAGG | |
| | | | CACGGTGTGAGTGCCGCCTGGAGGTGCGA | |
| | | | GTGCCTCAGTGACCAGGCTGGCTCCTG | |
| | | | GGGATGGCCAGGTACAACCGGATGCCAG | |
| | | | CCCCGTGCCAGGAGCCTGGAGGGAAGTTG | |
| | | | GGGAAACCCCTCCCTACTGTTGGATGTAT | |
| | | | GTGTGACAAGTGTGTCTCCTGTGCTGCGA | |
| | | | TGGGGGATCAGCAGGGCAGTTGTCGGGC | |
| | | | AGTCCTGAGTGGGTGTTGCACAGACTGGT | |
| | | | CCACAGGGCTCCTGAAGGAAGCCCCTGGA | |
| | | | TCTTTGGGGTAAAAGGAGGGTGGCCTCAA | |
| | | | GAAACAATGTCTGGGGACAGGCCTTTCTG | |
| | | | GCCTGCTATGTCTTCCCAATGTTTATTGGG | |
| | | | CAATAAAAGATAAGTGCAGTCACAGAGA | |
| | | | ACTCACTCTTC | |
| 29 | SS101 | Human Δexon 33 | GAGTGGTTCACCGTCTTGGAGCATTACCG | Exon 31/ |
| | | | CCGCACCCACTGCGTGGTGCCAGAGCTCA | Exon 32/ |
| | | | TCATTGGCAATGGCTACTACTTCCGCGTCT | Exon 34/ |
| | | | TCAGCCAGAATATGGTTGGCTTTAGTGAC | Exon 35 |
| | | | AGAGCGGCCACCACCAAGGAGCCCGTCTT | |
| | | | TATCCCCAGACCAGGCATCACCTATGAG | |
| | | | CCACCCAACTATAAGGCCCTGGACTTC | |
| | | | TCCGAGGCCCCAAGCTTCACCCAGCCC | |
| | | | CTGGTGAACCGCTCGGTCATCGCGGGC | |
| | | | TACACTGCTATGCTCTGCTGTGCTGTCC | |
| | | | GGGGTAGCCCCAAGTGCCTCAGTGACCA | |
| | | | GGCTGGCTCCTGGGGATGGCCAGGTACA | |
| | | | ACCGGATGCCAGCCCCGTGCCAGGAGC | |
| | | | CTGGAGGGAAGTTGGGGAAACCCCTCC | |
| | | | CTACTGTTGGATGTATGTGTGACAAGT | |
| | | | GTGTCTCCTGTGCTGCGATGGGGGATC | |
| | | | AGCAGGGCAGTTGTCGGGCAGTCCTGA | |
| | | | GTGGGTGTTGCACAGACTGGTCCACAG | |
| | | | GGCTCCTGAAGGAAGCCCCTGGATCTT | |
| | | | TGGGGTAAAAGGAGGGTGGCCTCAAGA | |
| | | | AACAATGTCTGGGGACAGGCCTTTCTG | |
| | | | GCCTGCTATGTCTTCCCAATGTTTATTG | |
| | | | GGCAATAAAAGATAAGTGCAGTCACAG | |
| | | | AGAACTCACTCTTC | |
| 30 | SS401 | Human Δexon 33 | GAGTGGTTCACCGTCTTGGAGCATTACCG | Exon 31/ |
| | | alt splice | CCGCACCCACTGCGTGGTGCCAGAGCTCA | Exon 32/ |
| | | | TCATTGGCAATGGCTACTACTTCCGCGTCT | Exon 33 alts/ |
| | | | TCAGCCAGAATATGGTTGGCTTTAGTGAC | Exon 34/ |
| | | | AGAGCGGCCACCACCAAGGAGCCCGTCTT | Exon 35 |
| | | | TATCCCCAGACCAGGCATCACCTATGAG | |
| | | | CCACCCAACTATAAGGCCCTGGACTTC | |
| | | | TCCGAGGCCCCAAGCTTCACCCAGCCC | |
| | | | CTGGTGAACCGCTCGGTCATCGCGGGC | |
| | | | TACACTGCTATGCTCTGCTGTGCTGTCC | |
| | | | GGGGTAGCCCCAAGGGCCACCAACTTAC | |
| | | | AGGGCGAGGCACGGTGTGAGTGCCGCCT | |
| | | | GGAGGTGCGAGTGCCTCAGTGACCAGG | |
| | | | CTGGCTCCTGGGGATGGCCAGGTACAA | |
| | | | CCGGATGCCAGCCCCGTGCCAGGAGCCTG | |
| | | | GAGGGAAGTTGGGGAAACCCCTCCCTACT | |
| | | | GTTGGATGTATGTGTGACAAGTGTGTCTC | |
| | | | CTGTGCTGCGATGGGGGATCAGCAGGGCA | |
| | | | GTTGTCGGGCAGTCCTGAGTGGGTGTTGC | |
| | | | ACAGACTGGTCCACAGGGCTCCTGAAGGA | |
| | | | AGCCCCTGGATCTTTGGGGTAAAAGGAGG | |
| | | | GTGGCCTCAAGAAACAATGTCTGGGGACA | |
| | | | GGCCTTTCTGGCCTGCTATGTCTTCCCAAT | |
| | | | GTTTATTGGGCAATAAAAGATAAGTGCAG | |
| | | | TCACAGAGAACTCACTCTTC | |
| 31 | SS201 | Mouse WT | GAGTGGTTCACGGTTTTGGAACACTACCG | Exon 31/ |
| | | | ACGCACTCACTGTGTGGTATCAGAGCTTA | Exon 32/ |
| | | | TCATTGGCAATGGCTACTACTTCCGGGTC | Exon 33/ |
| | | | TTCAGCCATAACATGGTGGGTTCCAGTGA | Exon 34/ |
| | | | CAAAGCTGCCGCCACCAAGGAGCCAGTCT | Exon 35 |
| | | | TTATTCCAAGACCAGGCATCACATATGA | |
| | | | GCCACCCAAATACAAGGCCCTGGACTT | |
| | | | CTCTGAGGCCCCAAGCTTCACCCAGCC | |
| | | | CTTGGCAAATCGCTCCATCATTGCAGG | |
| | | | CTATAATGCCATCCTCTGCTGTGCTGTC | |
| | | | CGAGGTAGTCCTAAGCCCAAGATTTCCT | |
| | | | GGTTCAAGAATGGCCTGGATCTGGGAGAA | |
| | | | GATGCTCGCTTCCGCATGTTCTGCAAGCA | |
| | | | GGGAGTATTGACCCTGGAGATCAGGAAA | |
| | | | CCCTGCCCCTATGATGGTGGTGTCTATGTC | |
| | | | TGCAGGGCCACCAACTTGCAGGGCGAGG | |
| | | | CACAGTGTGAGTGCCGCCTGGAGGTGCGA | |
| | | | GTTCCTCAGTGACCAGGATGGCTCCCC | |
| | | | AGAGATGGCTAGGTACAAATGGATGCCA | |
| | | | GGCTGTGTACCAGACCGGAAGGGAGTTG | |
| | | | GAGGAGCACCCTTTTCTGCTACTGCATGT | |
| | | | GTGTGTGCAACTGTGCATCCTGGAAGGAC | |
| | | | TGGCCAGCAGTGACACCAGGCAGGTCTGC | |
| | | | TGGGTTCTGAAGAAACTGACCCTAAGGAT | |
| | | | AATGTTAATACTGGGAGCATAAAGTGTGT | |
| | | | GGGCTTCAGAAGTGGTGACTGGGGACAG | |
| | | | GCCCTTTTGGCTGGCTCATTGTGTAGGAG | |
| | | | CAATAAAAGATCTGTGCCATTCCTGGGG | |
| 32 | SS301 | Mouse Δexon 33 | GAGTGGTTCACGGTTTTGGAACACTACCG | Exon 31/ |
| | | | ACGCACTCACTGTGTGGTATCAGAGCTTA | Exon 32/ |
| | | | TCATTGGCAATGGCTACTACTTCCGGGTC | Exon 34/ |
| | | | TTCAGCCATAACATGGTGGGTTCCAGTGA | Exon 35 |
| | | | CAAAGCTGCCGCCACCAAGGAGCCAGTCT | |
| | | | TTATTCCAAGACCAGGCATCACATATGA | |
| | | | GCCACCCAAATACAAGGCCCTGGACTT | |
| | | | CTCTGAGGCCCCAAGCTTCACCCAGCC | |
| | | | CTTGGCAAATCGCTCCATCATTGCAGG | |
| | | | CTATAATGCCATCCTCTGCTGTGCTGTC | |
| | | | CGAGGTAGTCCTAAGTTCCTCAGTGACC | |
| | | | AGGATGGCTCCCCAGAGATGGCTAGGTA | |
| | | | CAAATGGATGCCAGGCTGTGTACCAGA | |
| | | | CCGGAAGGGAGTTGGAGGAGCACCCTT | |
| | | | TTCTGCTACTGCATGTGTGTGTGCAACT | |
| | | | GTGCATCCTGGAAGGACTGGCCAGCAG | |
| | | | TGACACCAGGCAGGTCTGCTGGGTTCT | |
| | | | GAAGAAACTGACCCTAAGGATAATGTT | |
| | | | AATACTGGGAGCATAAAGTGTGTGGGC | |
| | | | TTCAGAAGTGGTGACTGGGGACAGGCC | |
| | | | CTTTTGGCTGGCTCATTGTGTAGGAGC | |
| | | | AATAAAAGATCTGTGCCATTCCTGGGG | |
| 33 | SS501 | Mouse Δexon 33 | GAGTGGTTCACGGTTTTGGAACACTACCG | Exon 31/ |
| | | alt splice | ACGCACTCACTGTGTGGTATCAGAGCTTA | Exon 32/ |
| | | | TCATTGGCAATGGCTACTACTTCCGGGTC | Exon 33 alts/ |
| | | | TTCAGCCATAACATGGTGGGTTCCAGTGA | Exon 34/ |
| | | | CAAAGCTGCCGCCACCAAGGAGCCAGTCT | Exon 35 |
| | | | TTATTCCAAGACCAGGCATCACATATGA | |
| | | | GCCACCCAAATACAAGGCCCTGGACTT | |
| | | | CTCTGAGGCCCCAAGCTTCACCCAGCC | |
| | | | CTTGGCAAATCGCTCCATCATTGCAGG | |
| | | | CTATAATGCCATCCTCTGCTGTGCTGTC | |
| | | | CGAGGTAGTCCTAAGGGCCACCAACTTG | |
| | | | CAGGGCGAGGCACAGTGTGAGTGCCGCCT | |
| | | | GGAGGTGCGAGTTCCTCAGTGACCAGGA | |
| | | | TGGCTCCCCAGAGATGGCTAGGTACAA | |
| | | | ATGGATGCCAGGCTGTGTACCAGACCGGA | |
| | | | AGGGAGTTGGAGGAGCACCCTTTTCTGCT | |
| | | | ACTGCATGTGTGTGTGCAACTGTGCATCC | |
| | | | TGGAAGGACTGGCCAGCAGTGACACCAG | |
| | | | GCAGGTCTGCTGGGTTCTGAAGAAACTGA | |
| | | | CCCTAAGGATAATGTTAATACTGGGAGCA | |
| | | | TAAAGTGTGTGGGCTTCAGAAGTGGTGAC | |
| | | | TGGGGACAGGCCCTTTTGGCTGGCTCATT | |
| | | | GTGTAGGAGCAATAAAAGATCTGTGCCAT | |
| | | | TCCTGGGG | |
| 34 | SS000 | Control | TGCATCTCATTGTAG | Scrambled |

### Pharmaceutical Compositions

In another embodiment, a pharmaceutical composition is provided, comprising an effective amount of an antisense oligonucleotide that specifically and selectively targets a mutant mRNA transcript of an MYBPC3 allele comprising a 25 bp deletion (MYBPC3^{Δ25bp}); and at least one pharmaceutically acceptable excipient.

The pharmaceutically acceptable excipient, or carrier, must be “acceptable” in the sense of being compatible with the other ingredients of the pharmaceutical composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for parenteral administration. In a specific embodiment, the compositions disclosed herein are suitable for parenteral or intramuscular administration, although other specific means of parenteral administration are also viable (such as, for example, intravenous, infusion, intra-arterial, or subcutaneous administration). The compositions may be prepared by any methods well known in the art of pharmacy, for example, using methods such as those described in Remington: The Science and Practice of Pharmacy (23rd ed., Adeboye Adejare, ed., 2020, see Section 7: Pharmaceutical Materials and Devices/Industrial Pharmacy). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given pharmaceutical composition.

For parenteral administration, suitable compositions include aqueous and non-aqueous sterile suspensions for intramuscular and/or intravenous administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In embodiments, the pharmaceutical composition may be formulated for injection. In other embodiments, the pharmaceutical composition may be formulated for infusion. In a specific embodiment, the pharmaceutical composition is formulated for an intramuscular injection, for example, to the cardiac muscle.

The term “effective amount.” as used herein, refers to the amount of a composition that is sufficient to achieve a desired biological effect. Generally, the dosage needed to provide an effective amount of the composition will vary depending upon such factors as the subject's age, condition, sex, and other variables which can be adjusted by one of ordinary skill in the art. The compositions of the present disclosure can be administered by either single or multiple dosages of an effective amount. In a specific embodiment, the effective amount is an amount sufficient to modulate, downregulate, inhibit, or silence expression of a MYBPC3^{Δ25bp} variant in the cardiac muscle of a subject.

Antisense oligonucleotides may be delivered to a target cell via a variety of techniques, including viral vectors, lipid particles, plasmids, liposomes, polymers, nanocarriers, metallic nanoparticles, extracellular vesicles, micelles, and the like. Such techniques are known in the art and readily available to the skilled person. See, for example Xin, et al., Nano-based delivery of RNAi in cancer therapy. Molecular Cancer 16, 134 (2017); Huang, et al., Nonviral delivery systems for antisense oligonucleotide therapeutics. Biomaterials Research 26: article 49 (2022).

### Methods of Treating Cardiac Disorders

In another embodiment, a method of treating a cardiac disorder in a subject in need thereof is provided, the method comprising administering to the subject an effective amount of an antisense oligonucleotide according to any of the embodiments disclosed herein. In embodiment, the ASO targets a mutant mRNA transcript of an MYBPC3^{Δ25bp} allele. In embodiments, the MYBPC3^{Δ25bp} allele comprises a 25 bp deletion within intron 32 of MYBPC3.

In embodiments, the target mutant mRNA transcript of the MYBPC3^{Δ25bp} allele skips exon 33 of MYBPC3 or is a product of altered splicing of exon 33 of MYBPC3. In embodiments, the target the mutant mRNA transcript of the MYBPC3^{Δ25bp} allele comprises a mis-spliced, full-length exon 34 of MYBPC3.

In embodiments, the cardiac disorder is selected from the group consisting of hypertrophic cardiomyopathy, heart failure, heart failure with preserved ejection fraction, and combinations thereof.

In embodiments, the method reduces expression of a mutant cardiac myosin binding protein C having a C10 domain modification (cMyBP-C^{Δ10}).

In embodiments, the method further comprises administering to the subject an effective amount of a second therapeutic agent. In embodiments, the second therapeutic agent is an agent typically administered to treat the symptoms of HCM and/or heart failure. In embodiments, the second therapeutic agent is selected from the group consisting of an angiotensin-converting enzyme (ACE) inhibitor, an angiotensin II receptor blocker (ARB), a beta blocker, a calcium channel blocker, a diuretic, and combinations thereof.

In embodiments, the ASOs according to the present disclosure and the second active agent are co-administered. “Co-administered.” as used herein, refers to administration of the ASO and the second therapeutic agent such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the ASO and the second therapeutic agent may be administered concurrently or sequentially.

In another embodiment, method is provided for modulating splicing of MYBPC3 processed mRNA in a cell, the method comprising contacting the mRNA with an ASO according to any of the embodiments disclosed herein. In a specific embodiment, the ASO is selected from the oligonucleotides set forth in Table 1.

### EXAMPLES

The following examples are given to illustrate various features of the present disclosure and are not intended to be limiting.

### Example 1. Subjects Carrying the MYBPC3\25 bp Variant Show Increased Cardiac Function Under Baseline and Exercise Testing

Ten asymptomatic South Asian carriers of the MYBPC3^{Δ25bp} variant and ten age- and gender-matched non-carriers (NCs) were tested for detectable subclinical risk factors under exercise stress conditions using bicycle exercise echocardiography and continuous cardiac monitoring, likely predisposing this group to LV dysfunction. Baseline parameters were substantially the same between the two groups, but the estimated effect of stress and genotype showed significantly higher ejection fraction (%) in carriers compared to non-carriers (FIG. 3C). These data confirm that MYBPC3^{Δ25bp} confers a significant predisposition to developing HF.

### Example 2. Expression of cMyBP-C^{ΔC10} Results in Decreased Contractile Function in Cultured Cardiomyocytes In Vitro and Develops HCM in Mice

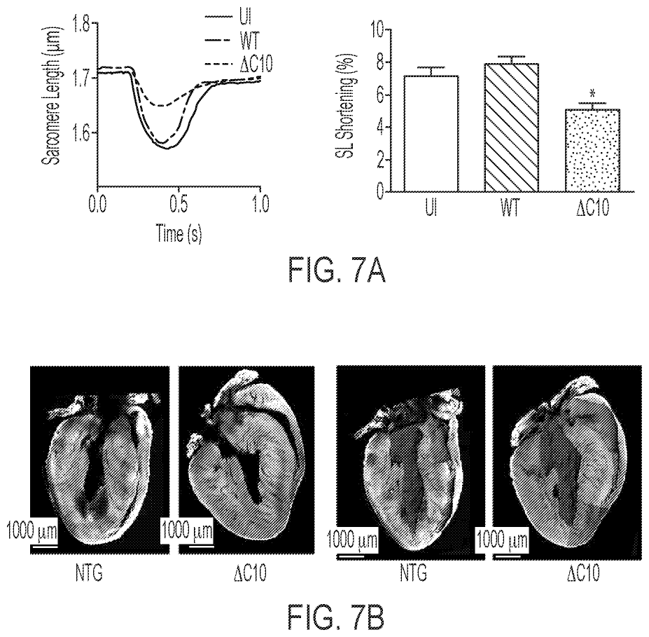

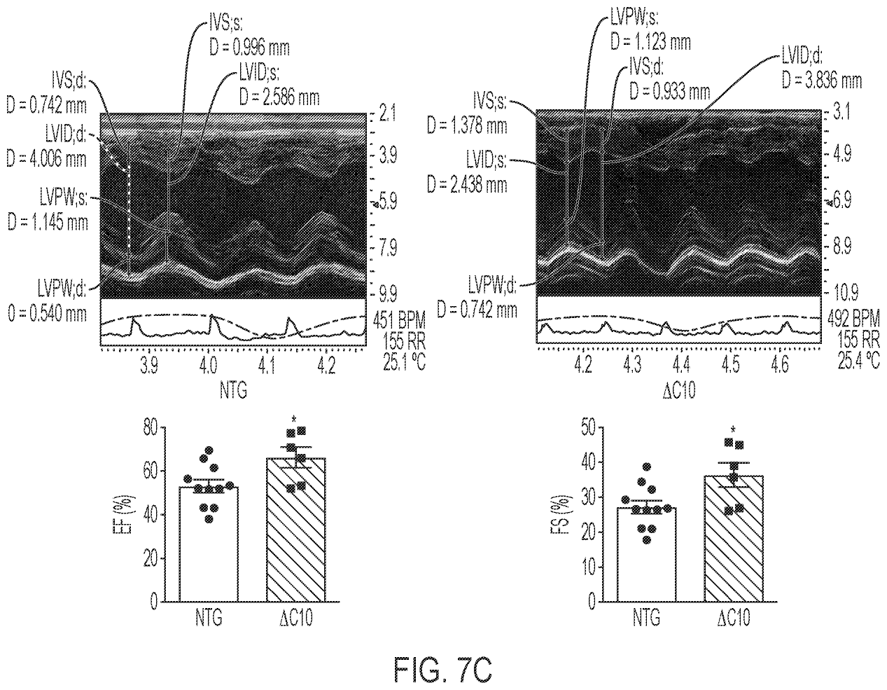

Next, the impact of the abnormal cMyBP-C^{ΔC10} protein was characterized. An adenovirus that expresses the cMyBP-C^{ΔC10} protein led to little or no localization to the C-zone in adult rat ventricular cardiomyocytes, whereas wild type cMyBP-C showed only C-zone staining, suggesting that cMyBP-C^{ΔC10} does not properly localize to the C-zone of the sarcomere (data not shown). Subcellular fractionation confirmed that most cMyBP-C^{ΔC10} resided in the soluble fraction with reduced presence in the myofilament fraction. cMyBP-C^{ΔC10} also displayed significantly reduced unloaded shortening velocity and relaxation velocity, which were attributed to defects in sarcomere function (FIG. 7A), as Ca²⁺ transients were unaffected. Together, these results show that the pathogenicity of cMyBP-C^{ΔC10} protein is sufficient to cause contractile dysfunction. Moreover, a transgenic mouse model expressing cMyBP-C^{ΔC10} showed increased LV free wall and septal thickening at 3 months of age, which confirmed a poison polypeptide effect (FIG. 7B). M-mode echocardiography of cMyBP-C^{ΔC10mut} (ΔC10) mice showed diastolic dysfunction with increased ejection fraction (EF, %), and fractional shortening (FS, %), compared to non-transgenic mice (NTG) (FIG. 7C).

Together, these results indicate that cMyBP-C^{ΔC10} protein could not properly incorporate in adult rat cardiomyocytes, causing a decrease in sarcomere contractility and suggesting a poison polypeptide effect.

### Example 3. MYBPC3^{Δ25bp} Heterozygous Mice Show an Alternative Transcript at Baseline, with Minimally Expressed Mutant mRNA

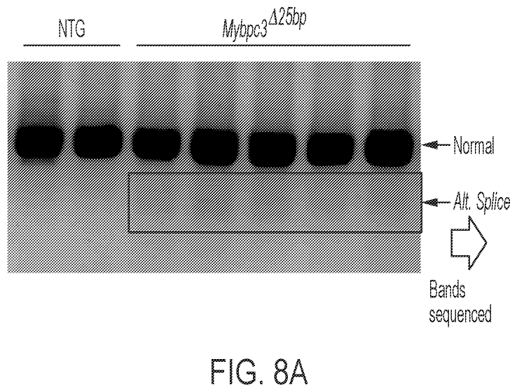

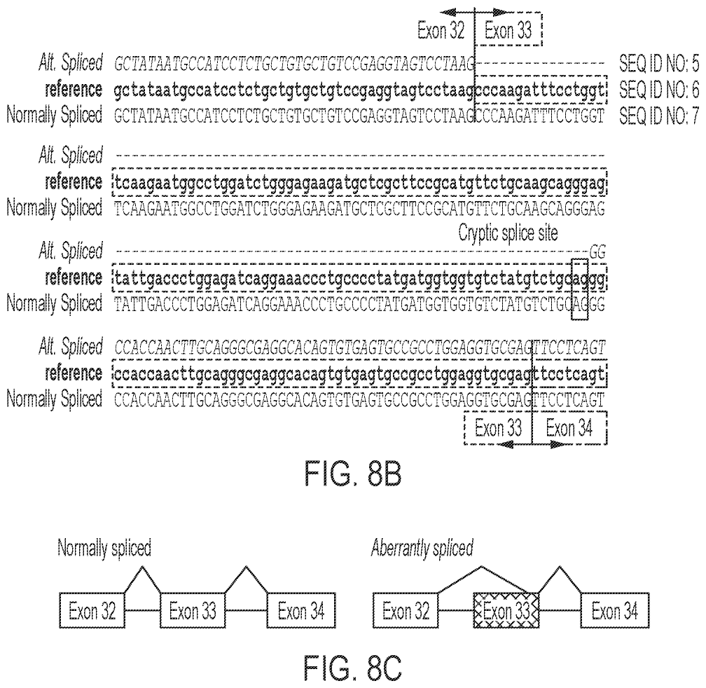

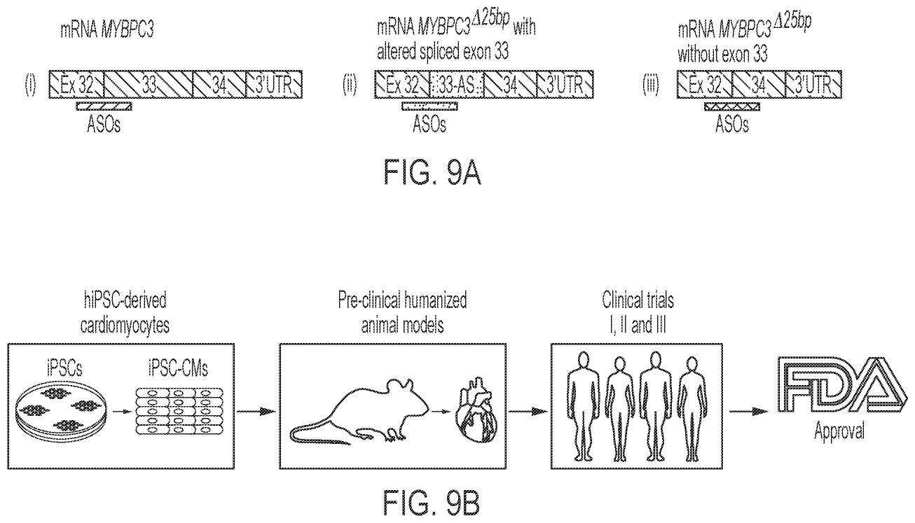

Using a MYBPC3^{Δ25bp} heterozygous mouse model, the presence of altered splicing in the middle of exon 33 was confirmed by RT-PCR experiments. Mouse cardiac tissue samples from transgenic and nontransgenic (NTG) control mice were obtained and analyzed via RT-PCR followed by agarose gel imaging. Results showed the presence of the altered splicing mRNA from MYBPC3^{Δ25bp} heterozygous mice, compared to NTG mouse hearts (FIG. 8A). DNA sequencing determined the exact region of altered splicing in exon 33 (FIG. 8B). The schematic diagram set forth in FIG. 8C illustrates exon 33 normal splicing (left side) and exon 33 altered splicing (right side). Based on the altered splicing. ASOs (SS401 and SS501. Table 1) were designed to specifically target mutant mRNA.

Aspects of the present disclosure can be described with reference to the following numbered clauses, with preferred features laid out in dependent clauses.
1. An antisense oligonucleotide that specifically and selectively targets a mutant mRNA transcript of a cardiac myosin binding protein C (MYBP (3) allele comprising a 25 bp deletion (MYBPC3^{Δ25bp}).
2. The antisense oligonucleotide according to clause 1, wherein the MYBPC3^{Δ25bp} allele comprises the 25 bp deletion within intron 32 of MYBPC3.
3. The antisense oligonucleotide according to any of the preceding clauses, wherein the mutant mRNA transcript of the MYBPC3^{Δ25bp} allele skips exon 33 of MYBPC3.
4. The antisense oligonucleotide according any of the preceding clauses, wherein the mutant mRNA transcript of the MYBPC3^{Δ25bp} allele is a product of altered splicing of exon 33 of MYBPC3.
5. The antisense oligonucleotide according to any of the preceding clauses, wherein the mutant mRNA transcript of the MYBPC3^{Δ25bp} allele comprises a full-length exon 34 of MYBPC3.
6. The antisense oligonucleotide according to any of the preceding clauses, wherein the mutant mRNA transcript of the MYBPC3^{Δ25bp} allele encodes a cMyBP-C protein comprising a mutant C10 domain (cMyBP-C^{C10mut}).
7. The antisense oligonucleotide according to any of the preceding clauses, wherein the MYBPC3^{Δ25bp} allele comprises SEQ ID NO: 4.
8. The antisense oligonucleotide according to any of the preceding clauses, wherein the antisense oligonucleotide hybridizes to SEQ ID NO: 4 or SEQ ID NO: 5, optionally under stringent conditions.
9. The antisense oligonucleotide according to any of the preceding clauses, having at least 85%, at least 90%, at least 95%, at least 96%, at least 97%, at least 98%, at least 99%, or 100% sequence identity with SEQ ID NO: 12. SEQ ID NO: 13, SEQ ID NO: 14, SEQ ID NO: 15. SEQ ID NO: 19. SEQ ID NO: 20, SEQ ID NO: 21. SEQ ID NO: 22, SEQ ID NO: 23, SEQ ID NO: 24. SEQ ID NO: 25. SEQ ID NO: 26, SEQ ID NO: 27, or any sequence set forth in Table 1.
10. The antisense oligonucleotide according to any of the preceding clauses, wherein the antisense oligonucleotide is complementary to a sequence having at least 85%, at least 90%, at least 95%, at least 96%, at least 97%, at least 98%, at least 99%, or 100% sequence identity with SEQ ID NO: 12, SEQ ID NO: 13, SEQ ID NO: 14, SEQ ID NO: 15, SEQ ID NO: 19, SEQ ID NO: 20. SEQ ID NO: 21, SEQ ID NO: 22. SEQ ID NO: 23. SEQ ID NO: 24, SEQ ID NO: 25, SEQ ID NO: 26. SEQ ID NO: 27, or any sequence set forth in Table 1.
11. The antisense oligonucleotide according to any of the preceding clauses, wherein the antisense oligonucleotide comprises at least one modification to a nucleotide or nucleoside sugar selected from 2′-O-methyl and 2′-O-methoxyethyl.
12. The antisense oligonucleotide according to any of the preceding clauses, wherein the antisense oligonucleotide comprises at least one modification selected from a morpholino substitution, a phosphorothioate linkage, and a phosphorodiamidate linkage.
13. A method of treating a cardiac disorder in a subject in need thereof, the method comprising administering to the subject an effective amount of an antisense oligonucleotide that targets a mutant mRNA transcript of an MYBPC3^{Δ25bp} allele.
14. The method according to clause 13, wherein the cardiac disorder is selected from hypertrophic cardiomyopathy, heart failure, and combinations thereof.
15. The method according to any of clauses 13-14, wherein the method reduces expression of a mutant cardiac myosin binding protein C having a C10 domain modification (cMyBP-C^{Δ10}).
16. The method according to any of clauses 13-15, wherein the MYBPC3^{Δ25bp} allele comprises a 25 bp deletion within intron 32 of MYBPC3.
17. The method according to any of clauses 13-16, wherein the mutant mRNA transcript of the MYBPC3^{Δ25bp} allele: skips exon 33 of MYBPC3; or is a product of altered splicing of exon 33 of MYBPC3.
18. The method according to any of clauses 13-17, wherein the mutant mRNA transcript of the MYBPC3^{Δ25bp} allele comprises a full-length exon 34 of MYBPC3.
19. The method according to any of clauses 13-18, wherein the MYBPC3^{Δ25bp} allele comprises SEQ ID NO: 4.
20. The method according to any of clauses 13-19, wherein the antisense oligonucleotide hybridizes to SEQ ID NO: 4 or SEQ ID NO: 5.
21. The method according to any of clauses 13-20, wherein the antisense oligonucleotide has at least 85%, at least 90%, at least 95%, at least 96%, at least 97%, at least 98%, at least 99%, or 100% sequence identity with SEQ ID NO: 12. SEQ ID NO: 13, SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25. SEQ ID NO: 26, SEQ ID NO: 27, or any sequence set forth in Table 1.
22. The method according to any of clauses 13-21, wherein the antisense oligonucleotide is complementary to a sequence having at least 85%, at least 90%, at least 95%, at least 96%, at least 97%, at least 98%, at least 99%, or 100% sequence identity with SEQ ID NO: 12. SEQ ID NO: 13, SEQ ID NO: 14, SEQ ID NO: 15, SEQ ID NO: 19. SEQ ID NO: 20, SEQ ID NO: 21. SEQ ID NO: 22, SEQ ID NO: 23, SEQ ID NO: 24, SEQ ID NO: 25, SEQ ID NO: 26, SEQ ID NO: 27, or any sequence set forth in Table 1.
23. The method according to any of clauses 13-22, wherein the antisense oligonucleotide comprises at least one modification to a nucleotide or nucleoside sugar selected from 2′-O-methyl and 2′-O-methoxyethyl.
24. The method according to any of clauses 13-23, wherein the antisense oligonucleotide comprises at least one modification selected from a morpholino substitution, a phosphorothioate linkage, and a phosphorodiamidate linkage.
25. A method of treating a cardiac disorder in a subject in need thereof, the method comprising administering to the subject an effective amount of the antisense oligonucleotide according to any of clauses 1-10.
26. The method according to clause 25, further comprising administering to the subject an effective amount of a second therapeutic agent.
27. The method according to clause 26, wherein the second therapeutic agent is selected from the group consisting of an angiotensin-converting enzyme (ACE) inhibitor, an angiotensin II receptor blocker (ARB), a beta blocker, a calcium channel blocker, a diuretic, and combinations thereof.
28. A pharmaceutical composition comprising: an effective amount of the antisense oligonucleotide according to any of clauses 1-10; and at least one pharmaceutically acceptable excipient.
29. The pharmaceutical composition according to clause 28, wherein the antisense oligonucleotide is associated with a carrier selected from the group consisting of a lipid-based nanoparticle, a liposome, a polymer, a micelle, an extracellular vesicle, a plasmid, and a viral vector.
30. A method for modulating splicing of MYBPC3 processed mRNA in a cell, the method comprising contacting the mRNA with an antisense oligonucleotide as set forth in Table 1.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An antisense oligonucleotide that specifically and selectively targets a mutant mRNA transcript of a cardiac myosin binding protein C (MYBPC3) allele comprising a 25 bp deletion (MYBPC3^{Δ25bp}).

2. The antisense oligonucleotide according to claim 1, wherein the MYBPC3^{Δ25bp} allele comprises the 25 bp deletion within intron 32 of MYBPC3.

3. The antisense oligonucleotide according to claim 2, wherein the mutant mRNA transcript of the MYBPC3^{Δ25bp} allele skips exon 33 of MYBPC3.

4. The antisense oligonucleotide according to claim 2, wherein the mutant mRNA transcript of the MYBPC3^{Δ25bp} allele is a product of altered splicing of exon 33 of MYBPC3.

5. The antisense oligonucleotide according to claim 2, wherein the mutant mRNA transcript of the MYBPC3^{Δ25bp} allele comprises a full-length exon 34 of MYBPC3.

6. The antisense oligonucleotide according to claim 1, wherein the mutant mRNA transcript of the MYBPC3^{Δ25bp} allele encodes a cMyBP-C protein comprising a mutant C10 domain (cMyBP-C^{C10mut}).

7. The antisense oligonucleotide according to claim 1, wherein the MYBPC3^{Δ25bp} allele comprises SEQ ID NO: 4.

8. The antisense oligonucleotide according to claim 1, wherein the antisense oligonucleotide hybridizes to SEQ ID NO: 4 or SEQ ID NO: 5.

9. The antisense oligonucleotide according to claim 1, wherein the antisense oligonucleotide has or is complementary to a sequence having at least 85%, at least 90%, at least 95%, at least 96%, at least 97%, at least 98%, at least 99%, or 100% sequence identity with SEQ ID NO: 12, SEQ ID NO: 13, SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

10. (canceled)

11. The antisense oligonucleotide according to claim 1, wherein the antisense oligonucleotide comprises at least one modification to a nucleotide sugar selected from 2′-O-methyl and 2′-O-methoxyethyl.

12. The antisense oligonucleotide according to claim 1, wherein the antisense oligonucleotide comprises at least one modification selected from a morpholino substitution, a phosphorothioate linkage, and a phosphorodiamidate linkage.

13. A method of treating a cardiac disorder in a subject in need thereof, the method comprising administering to the subject an effective amount of an antisense oligonucleotide that targets a mutant mRNA transcript of an MYBPC3^{Δ25bp} allele.

14. The method according to claim 13, wherein the cardiac disorder is selected from hypertrophic cardiomyopathy, heart failure, and combinations thereof.

15. The method according to claim 13, wherein the method reduces expression of a mutant cardiac myosin binding protein C having a C10 domain modification (cMyBP-C^{Δ10}).

16. The method according to claim 13, wherein the MYBPC3^{Δ25bp} allele comprises a 25 bp deletion within intron 32 of MYBPC3.

17. The method according to claim 13, wherein the mutant mRNA transcript of the MYBPC3^{Δ25bp} allele:
skips exon 33 of MYBPC3; or
is a product of altered splicing of exon 33 of MYBPC3.

18. The method according to claim 13, wherein the mutant mRNA transcript of the MYBPC3^{Δ25bp} allele comprises a full-length exon 34 of MYBPC3.

19. The method according to claim 13, wherein the MYBPC3^{Δ25bp} allele comprises SEQ ID NO: 4.

20. The method according to claim 13, wherein the antisense oligonucleotide hybridizes to SEQ ID NO: 4 or SEQ ID NO: 5.

21. The method according to claim 13, wherein the antisense oligonucleotide has or is complementary to a sequence having at least 85%, at least 90%, at least 95%, at least 96%, at least 97%, at least 98%, at least 99%, or 100% sequence identity with SEQ ID NO: 12, SEQ ID NO: 13, SEQ ID NO: 14, SEQ ID NO: 15, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

22. (canceled)

23. The method according to claim 13, wherein the antisense oligonucleotide comprises at least one modification to a nucleotide sugar selected from 2′-O-methyl and 2′-O-methoxyethyl.

24. The method according to claim 13, wherein the antisense oligonucleotide comprises at least one modification selected from a morpholino substitution, a phosphorothioate linkage, and a phosphorodiamidate linkage.

25-30. (canceled)


SEQUENCE LISTING (34 sequences)

SEQ ID NO: 1 - protein, 94 aa, Homo sapiens
PSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 2 - protein, 87 aa, Homo sapiens
PSFTQPLVNRSVIAGYTAMLCCAVRGSPKCLSDQAGSWGWPGTTGCQPRARSLEGSWGNPSLLLDVCVTSVSPVLRWGISRAVVGQS

SEQ ID NO: 3 - DNA, 53 bp, Homo sapiens
  source 1..53: mol_type = genomic DNA
aggtcccctctcagcctggatggcttccctccctctctttaccttatttatag

SEQ ID NO: 4 - DNA, 28 bp, Homo sapiens
  source 1..28: mol_type = genomic DNA
aggtcccctctctttaccttatttatag

SEQ ID NO: 5 - DNA, 106 bp, Homo sapiens
  source 1..106: mol_type = genomic DNA
gctataatgccatcctctgctgtgctgtccgaggtagtcctaagggccaccaacttgcagggcgaggcacagtgtgagtgccgcctggaggtgcgagttcctcagt

SEQ ID NO: 6 - DNA, 239 bp, Homo sapiens
  source 1..239: mol_type = genomic DNA
gctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaactgcagggcgaggcacagtgtgagtgccgcctggaggtgcgagttcctcagt

SEQ ID NO: 7 - DNA, 239 bp, Homo sapiens; identical to SEQ ID NO: 6
  source 1..239: mol_type = genomic DNA

SEQ ID NO: 8 - DNA, 20 bp, synthetic construct
  source 1..20: mol_type = other DNA
aggaaatcttgggcttgggg

SEQ ID NO: 9 - DNA, 20 bp, synthetic construct
  source 1..20: mol_type = other DNA
gaaatcttgggcttggggct

SEQ ID NO: 10 - DNA, 20 bp, synthetic construct
  source 1..20: mol_type = other DNA
aatcttgggcttggggctac

SEQ ID NO: 11 - DNA, 18 bp, synthetic construct
  source 1..18: mol_type = other DNA
atcttgggcttggggcta

SEQ ID NO: 12 - DNA, 19 bp, synthetic construct
  source 1..19: mol_type = other DNA
actgaggcacttggggtac

SEQ ID NO: 13 - DNA, 20 bp, synthetic construct
  source 1..20: mol_type = other DNA
cactgaggcacttggggcta

SEQ ID NO: 14 - DNA, 20 bp, synthetic construct
  source 1..20: mol_type = other DNA
tcactgaggcacttggggct

SEQ ID NO: 15 - DNA, 18 bp, synthetic construct
  source 1..18: mol_type = other DNA
gtcactgaggcacttggg

SEQ ID NO: 16 - DNA, 20 bp, synthetic construct
  source 1..20: mol_type = other DNA
gaaatcttgggcttaggact

SEQ ID NO: 17 - DNA, 20 bp, synthetic construct
  source 1..20: mol_type = other DNA
aaatcttgggcttaggacta

SEQ ID NO: 18 - DNA, 20 bp, synthetic construct
  source 1..20: mol_type = other DNA
aatcttgggcttaggactac

SEQ ID NO: 19 - DNA, 20 bp, synthetic construct
  source 1..20: mol_type = other DNA
actgaggaacttaggactac

SEQ ID NO: 20 - DNA, 20 bp, synthetic construct
  source 1..20: mol_type = other DNA
cactgaggaacttaggacta

SEQ ID NO: 21 - DNA, 20 bp, synthetic construct
  source 1..20: mol_type = other DNA
tcactgaggaacttaggact

SEQ ID NO: 22 - DNA, 20 bp, synthetic construct
  source 1..20: mol_type = other DNA
aagttggtggcccttggggc

SEQ ID NO: 23 - DNA, 20 bp, synthetic construct
  source 1..20: mol_type = other DNA
agttggtggcccttggggct

SEQ ID NO: 24 - DNA, 20 bp, synthetic construct
  source 1..20: mol_type = other DNA
gttggtggcccttggggcta

SEQ ID NO: 25 - DNA, 20 bp, synthetic construct
  source 1..20: mol_type = other DNA
gttggtggcccttaggacta

SEQ ID NO: 26 - DNA, 20 bp, synthetic construct
  source 1..20: mol_type = other DNA
agttggtggcccttaggact

SEQ ID NO: 27 - DNA, 20 bp, synthetic construct
  source 1..20: mol_type = other DNA
aagttggtggcccttaggac

SEQ ID NO: 28 - RNA, 838 nt, Homo sapiens
  source 1..838: mol_type = mRNA
gagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttc

SEQ ID NO: 29 - RNA, 651 nt, Homo sapiens
  source 1..651: mol_type = mRNA
gagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttc

SEQ ID NO: 30 - RNA, 704 nt, Homo sapiens
  source 1..704: mol_type = mRNA
gagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactcactcttc

SEQ ID NO: 31 - RNA, 796 nt, Mus musculus
  source 1..796: mol_type = mRNA
gagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaacttgcagggcgaggcacagtgtgagtgccgcctggaggtgcgagttcctcagtgaccaggatggctccccagagatggctaggtacaaatggatgccaggctgtgtaccagaccggaagggagttggaggagcacccttttctgctactgcatgtgtgtgtgcaactgtgcatcctggaaggactggccagcagtgacaccaggcaggtctgctgggttctgaagaaactgaccctaaggataatgttaatactgggagcataaagtgtgtgggcttcagaagtggtgactggggacaggcccttttggctggctcattgtgtaggagcaataaaagatctgtgccattcctgggg

SEQ ID NO: 32 - RNA, 609 nt, Mus musculus
  source 1..609: mol_type = mRNA
gagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagttcctcagtgaccaggatggctccccagagatggctaggtacaaatggatgccaggctgtgtaccagaccggaagggagttggaggagcacccttttctgctactgcatgtgtgtgtgcaactgtgcatcctggaaggactggccagcagtgacaccaggcaggtctgctgggttctgaagaaactgaccctaaggataatgttaatactgggagcataaagtgtgtgggcttcagaagtggtgactggggacaggcccttttggctggctcattgtgtaggagcaataaaagatctgtgccattcctgggg

SEQ ID NO: 33 - RNA, 662 nt, Mus musculus
  source 1..662: mol_type = mRNA
gagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagggccaccaacttgcagggcgaggcacagtgtgagtgccgcctggaggtgcgagttcctcagtgaccaggatggctccccagagatggctaggtacaaatggatgccaggctgtgtaccagaccggaagggagttggaggagcacccttttctgctactgcatgtgtgtgtgcaactgtgcatcctggaaggactggccagcagtgacaccaggcaggtctgctgggttctgaagaaactgaccctaaggataatgttaatactgggagcataaagtgtgtgggcttcagaagtggtgactggggacaggcccttttggctggctcattgtgtaggagcaataaaagatctgtgccattcctgggg

SEQ ID NO: 34 - DNA, 15 bp, synthetic construct
  source 1..15: mol_type = other DNA
tgcatctcattgtag